(19) United States
(12) Patent Application Publication
(10) Pub. No.: US 2011/0059081 A1
(43) Pub. Date: Mar. 10, 2011

(54) METHODS AND COMPOSITIONS FOR THE TREATMENT OF RECEPTOR TYROSINE KINASE MEDIATED DISEASES OR DISORDERS

(75) Inventor: Sarah Bacus, Hinsdale, IL (US)

(73) Assignee: QUINTILES TRANSNATIONAL CORP., Durham, NC (US)

(21) Appl. No.: 12/878,163

(22) Filed: Sep. 9, 2010

Related U.S. Application Data
(60) Provisional application No. 61/240,785, filed on Sep. 9, 2009.

(51) Int. Cl. A61K 39/395; C12N 5/00; A61P 35/00

(52) U.S. Cl. 424/133.1; 435/375; 424/172.1; 424/142.1

(57) ABSTRACT

The present disclosure provides methods and compositions for treating a disease or disorder in a subject, the method comprising, administering to the subject a therapeutically effective amount of one or more receptor tyrosine kinase inhibitors and a therapeutically effective amount of one or more inhibitors of the dihydrofolate reductase (DHFR) pathway including, for example, methyltransferase inhibitors.

### BACKGROUND

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand TGF-α, is frequently associated with many cancers, including breast, lung, colorectal, ovarian, renal cell, bladder, head and neck cancers, glioblastomas, and astrocytomas. Activation of EGFR stimulated signaling pathways promote multiple processes that are potentially cancer-promoting, e.g. proliferation, angiogenesis, cell motility and invasion, decreased apoptosis and induction of drug resistance.

Several EGFR and Her family antagonists have been shown to offer clinical benefit, including erlotinib (OSI Pharmaceuticals/Genetech/Roche), gefitinib (Astra Zeneca) and lapatinib (GlaxoSmithKline). Anti-EGFR antibodies have also shown clinical utility, including cetuximab (Imclone/Bristol Myers) and panitumamab (Abgenix/Amgen) which are approved for the treatment of EGFR-expressing, metastatic colorectal carcinoma. A breakthrough in the field of EGFR-targeted therapy occurred in 2004 with the identification of somatic mutations in the EGFR gene, which were closely associated with a favorable clinical response to gefitinib and erlotinib treatment in NSCLC patients. These genetic alterations consisted of small in-frame deletions or point mutations in EGFR exons 18-24, which encode the kinase domain of the protein and are clustered in two mutational ‘hot spots’ in the EGFR gene.

Biomarkers have been employed to determine those patients that are most likely to respond to a particular therapy, including therapies directed to EGFR. For example, the presence of K-Ras mutations is associated with a lack of treatment response to gefitinib and erlotinib. Therefore, the presence of a K-Ras gene mutation has been used as a marker for selecting those patients who will not benefit from anti-EGFR therapy. Accordingly, therapeutic regimens that target EGFR may not be administered to patients predicted to be unresponsive to the regimen.

### SUMMARY

The present disclosure provides methods and compositions for treating a disease or disorder in a subject (e.g., a subject with one or more mutation in Ras). Such methods may comprise, administering to the subject a therapeutically effective amount of one or more receptor tyrosine kinase inhibitors (e.g., EGFR inhibitors) and a therapeutically effective amount of one or more inhibitors of the dihydrofolate reductase (DHFR) pathway including, for example, methyltransferase inhibitors.

The present disclosure provides methods and compositions for increasing the efficacy of treatment of a patient harboring a tumor with RAS mutation by mislocating RAS in the tumor cells from the cell membrane to the cytoplasm.

The present disclosure also provides methods for sensitizing (e.g., reducing the resistance) and treating a cell harboring a Ras mutation by contacting the cell with a therapeutically effective amount of one or more inhibitors of the dihydrofolate reductase (DHFR) pathway; and contacting the cell with a therapeutically effective amount of one or more receptor tyrosine kinase inhibitors.

The present disclosure also provides methods for treating a subject with a disease or disorder by obtaining a biological sample from the subject; assaying the biological sample for one or more Ras mutations; determining if one or more Ras mutations are present in the biological sample; and administering to the subject one or more inhibitors of the dihydrofolate reductase (DHFR) pathway including, for example, methyltransferase inhibitors where one or more Ras mutations are present in the biological sample and administering to the subject one or more receptor tyrosine kinase inhibitors where Ras mutations are not present in the biological sample.

The present disclosure also provides a pharmaceutical composition comprising a therapeutically effective amount of one or more receptor tyrosine kinase inhibitors and a therapeutically effective amount of one or more inhibitors of the dihydrofolate reductase (DHFR) pathway including, for example, methyltransferase inhibitors.

In an embodiment of any of the above-described methods and compositions, the methyltransferase inhibitor is Methotrexate.

In an embodiment of any of the above-described methods and compositions, the disease or disorder is characterized by one or more Ras mutations that lead to an oncogenic phenotype. In an embodiment of any of the above-described methods and compositions, Ras is k-Ras (as represented by SEQ ID NO: 1), n-Ras (as represented by SEQ ID NO: 2), or h-Ras (as represented by SEQ ID NO: 3). In an embodiment of any of the above-described methods and compositions, the k-Ras mutations in SEQ ID NO: 1 are mutations at position 12, 13 and/or 61. In an embodiment of any of the above-described methods and compositions, the k-Ras mutations are G12A, G12N, G12R, G12C, G12S, G12V, G13N and/or Q61H. In an embodiment of any of the above-described methods and compositions, the h-Ras or n-Ras mutations in SEQ ID NO: 2 or 3 are at positions 12, 13 and/or 61.

In an embodiment of any of the above-described methods and compositions, the receptor tyrosine kinase inhibitor is an antibody. In an embodiment of any of the above-described methods and compositions, the tyrosine kinase inhibitor targets HER1 (EGFR), HER2/neu, HER3, or any combination thereof.

In an embodiment of any of the above-described methods and compositions, the antibody is a monoclonal antibody. In an embodiment of any of the above-described methods and compositions, the monoclonal antibody is cetuximab (Erbitux), panitumumab zalutumumab, nimotuzumab or matuzumab.

In an embodiment of any of the above-described methods and compositions, the receptor tyrosine kinase inhibitor is a small molecule inhibitor. In an embodiment of any of the above-described methods and compositions, the small molecule inhibitor is gefitinib, erlotinib or lapatinib.

In an embodiment of any of the above-described methods and compositions, the therapeutically effective amount of one or more tyrosine kinase inhibitors and the therapeutically effective amount of one or more inhibitors of the dihydrofolate reductase (DHFR) pathway including, for example, methyltransferase inhibitors are optionally adapted for a co-treatment with radiotherapy or radio-immunotherapy.

In an embodiment of any of the above-described methods and compositions, the disease or disorder is cancer. In an embodiment of any of the above-described methods and compositions, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In an embodiment of any of the above-described methods and compositions, the subject is a cancer patient.

In an embodiment of any of the above-described methods and compositions, the biological sample is assayed for Ras mutations by analyzing nucleic acid obtained from the sample. In some embodiments, the biological sample is assayed for Ras mutations by analyzing proteins obtained from the sample.

In an embodiment of any of the above-described methods and compositions, the biological sample is a tumor biopsy. In an embodiment of any of the above-described methods and compositions, the biological sample is an aspirate.

### BRIEF DESCRIPTION OF THE DRAWINGS

FIG. 1 shows immunofluorescence staining of A549 lung tumor cells treated for 48 hours with 1.0 μM Methotrexate for RAS.

FIG. 2 shows immunofluorescence staining of A549 lung tumor cells treated for 48 hours with 1.0M Methotrexate for RAS.

FIG. 3 shows a Western blot for pERK1/2 expression in A549 cells treated with EGF, Methotrexate or a combination of EGF and Methotrexate.

FIG. 4 shows a Western blot for pAKT and pERK1/2 expression in A549 cells treated with EGF, Methotrexate, C225 antibody (an anti-EGFR antibody) or a combination of EGF, Methotrexate and C225 antibody.

### DETAILED DESCRIPTION

The Ras proto-oncogenes (e.g., H-ras, K-ras, and N-ras) encode approximately 21 kDa GTP-binding proteins which are involved in the EGFR signaling pathway. Several recent clinical studies have shown that the presence of a Ras mutation, such as K-Ras, is a significant predictor of resistance to treatment with receptor tyrosine kinase inhibitors including, for example, EGFR inhibitors. Cells that harbor activating mutations in Ras are believed to exhibit increased MAPK signaling as compared to cells with wild type Ras. As such, patients with Ras mutations (e.g., activating mutations in RAS) are often not treated with receptor tyrosine kinase inhibitors. The inventor has demonstrated that by preventing mutated Ras from being translocated and inserted into a cell's plasma membrane where it can affect MAPK signaling (e.g., by treatment with one or more inhibitors of the DHFR pathway such as Methotrexate), the cell becomes responsive to treatment with a receptor tyrosine kinase inhibitor. Surprisingly, it has been demonstrated that cells treated with a DHFR pathway inhibitor, such as Methotrexate, and an anti-EGFR antibody exhibit a synergistic decrease in MAPK signaling as compared to cells treated with either Methotrexate or an anti-EGFR antibody alone. Additionally, the methods may be used to treat patients (e.g., patients with Ras mutations) that are traditionally categorized as unresponsive to receptor tyrosine kinase inhibitors.

The present disclosure also provides methods for sensitizing (e.g., reducing the resistance of the cell to an EGFR inhibitor such as an EGFR antibody) and treating a cell harboring a Ras mutation by contacting the cell with a therapeutically effective amount of one or more inhibitors of the dihydrofolate reductase (DHFR) pathway (e.g., methotrexate). In some embodiments, the cell may be further contacted with a therapeutically effective amount of one or more receptor tyrosine kinase inhibitors.

The present disclosure provides methods and compositions for treating cancer by administering to a patient a therapeutically effective amount of one or more receptor tyrosine kinase inhibitors and a therapeutically effective amount of one or more inhibitors of the dihydrofolate reductase (DHFR) pathway including, for example, methyltransferase inhibitors (e.g., methotrexate).

The present disclosure also provides methods for treating a patient with a disease or disorder by obtaining a biological sample from the patient; assaying the biological sample for one or more Ras mutations; determining if one or more Ras mutations are present in the biological sample; and administering to the patient one or more receptor tyrosine kinase inhibitors and one or more inhibitors of the dihydrofolate reductase (DHFR) pathway including, for example, methyltransferase inhibitors where one or more Ras mutations are present in the biological sample and administering to the patient one or more receptor tyrosine kinase inhibitors where Ras mutations are not present in the biological sample.

The present disclosure also provides a pharmaceutical composition comprising a therapeutically effective amount of one or more receptor tyrosine kinase inhibitors and a therapeutically effective amount of one or more inhibitors of the dihydrofolate reductase (DHFR) pathway including, for example, methyltransferase inhibitors.

In one embodiment, the disease or disorder is cancer. In one embodiment the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment the cancer is non-small cell lung cancer, pancreatic cancer, breast cancer, ovarian cancer, colorectal cancer, or head and neck cancer.

In yet another embodiment the cancer is a carcinoma, a tumor, a neoplasm, a lymphoma, a melanoma, a glioma, a sarcoma, or a blastoma.

In one embodiment the carcinoma is selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment the tumor is selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment the neoplasm is selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma.

In a further embodiment the lymphoma is selected from the group consisting of: nervous system lymphoma, AIDS-related lymphoma, cutaneous T-cell lymphoma, non-Hodgkin's lymphoma, lymphoma, and Waldenstrom's macroglobulinemia.

In another embodiment the melanoma is selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In yet another embodiment the sarcoma is selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In one embodiment the glioma is selected from the group consisting of: glioma, brain stem glioma, and hypothalamic and visual pathway glioma.

In another embodiment the blastoma is selected from the group consisting of: pulmonary blastoma, pleuropulmonary blastoma, retinoblastoma, neuroblastoma, medulloblastoma, glioblastoma, and hemangiblastomas.

Proteins including, mutated Ras may detected in a biological sample by any known method in the art. Such methods may include but are not limited to Western blots, northern blots, Southern blots, ELISA, immunoprecipitation, immunofluorescence, flow cytometry, immunocytochemistry, nucleic acid hybridization techniques, nucleic acid reverse transcription methods, and nucleic acid amplification methods.

A biological sample may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, lymph fluid, urine and faeces, or tissue which has been removed from organs, such as breast, lung, intestine, skin, cervix, prostate, and stomach.

An exemplary method for detecting the presence or absence of a Ras protein or nucleic acid in a biological sample involves obtaining a biological sample (e.g. a tumor-associated body fluid) from a test subject and contacting the biological sample with a compound or an agent capable of detecting the polypeptide or nucleic acid (e.g., mRNA, genomic DNA, or cDNA). The detection methods of the disclosure can thus be used to detect mRNA, protein, cDNA, or genomic DNA, for example, in a biological sample in vitro as well as in vivo.

In an embodiment, the expression of Ras may be detected at the nucleic acid level. Nucleic acid-based techniques for assessing expression are well known in the art and include, for example, determining the level of Ras mRNA in a biological sample. Many expression detection methods use isolated RNA. Any RNA isolation technique that does not select against the isolation of mRNA can be utilized for the purification of RNA from cervical cells (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

Isolated mRNA from a biological sample can be used in hybridization or amplification assays that include, but are not limited to, Southern or Northern analyses, polymerase chain reaction analyses and probe arrays. One method for the detection of Ras mRNA levels involves contacting the isolated mRNA with a nucleic acid molecule (probe) that can hybridize to the mRNA encoded by the Ras gene. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding Ras. Hybridization of an mRNA with the probe indicates that Ras is being expressed.

In one embodiment, the mRNA from a biological sample is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array.

An alternative method for determining the level of Ras mRNA in a biological sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, biomarker expression may be assessed by quantitative fluorogenic RT-PCR (i.e., the TaqMan® System). Such methods typically may utilize pairs of oligonucleotide primers that are specific for Ras. Methods for designing oligonucleotide primers specific for a known sequence are well known in the art.

Expression levels of Ras RNA may be monitored using a membrane blot (such as used in hybridization analysis such as Northern, Southern, dot, and the like), or microwells, sample tubes, gels, beads or fibers (or any solid support comprising bound nucleic acids) (see, e.g., U.S. Pat. Nos. 5,770,722, 5,874,219, 5,744,305, 5,677,195 and 5,445,934). The detection of Ras expression may also comprise using nucleic acid probes in solution.

In one embodiment of the disclosure, microarrays are used to detect Ras expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA may be hybridized to complementary probes on the array and then detected by laser scanning. Hybridization intensities for each probe on the array are determined and converted to a quantitative value representing relative gene expression levels (see, e.g., U.S. Pat. Nos. 6,040,138, 5,800,992, 6,020,135, 6,033,860, and 6,344,316). High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device (see, e.g., U.S. Pat. Nos. 5,856,174 and 5,922,591).

In one approach, total mRNA isolated from the biological sample may be converted to labeled cRNA and then hybridized to an oligonucleotide array. Each sample may be hybridized to a separate array. Relative transcript levels may be calculated by reference to appropriate controls present on the array and in the sample.

In a particular embodiment, the level of Ras mRNA can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (see, e.g., U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of Ras mRNA levels involves contacting the isolated mRNA with a nucleic acid molecule (probe) that can hybridize to the Ras mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding Ras. Other suitable probes for use in the diagnostic assays of the disclosure are described herein. Hybridization of an mRNA with the probe indicates that Ras is being expressed.

In one format, the mRNA may be immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA may be contacted with the probe(s), for example, in an Affymetrix gene chip array. A skilled artisan can readily adapt known mRNA detection methods for use in detecting the level of mRNA encoded by Ras.

An alternative method for determining the level of Ras mRNA in a biological sample involves the process of nucleic acid amplification, e.g., by RT-PCR (see, e.g., U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample may be prepared/processed using known histological methods. The sample may be then immobilized on a support, typically a glass slide, and then contacted with a probe that can hybridize to Ras mRNA.

In another embodiment of the present disclosure, a Ras protein may be detected. A preferred agent for detecting Ras protein of the disclosure is an antibody capable of binding to such a protein or a fragment thereof, preferably an antibody with a detectable label. Antibodies can be polyclonal, or more preferably, monoclonal. An intact antibody, or a fragment or derivative thereof can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that may be directly labeled. Examples of indirect labeling include detection of a primary antibody using a fluorescently labeled secondary antibody and end-labeling of a DNA probe with biotin such that it can be detected with fluorescently labeled streptavidin.

Antibody fragments may comprise a portion of an intact antibody, preferably the antigen-binding or variable region of the intact antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and may be still capable of cross-linking antigen.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the disclosure, there also exist in the art, video-microscopy and software methods for the quantitative determination of an amount of multiple molecular species (e.g., biomarker proteins) in a biological sample wherein each molecular species present may be indicated by a representative dye marker having a specific color. Such methods are also known in the art as a colorimetric analysis methods. In these methods, video-microscopy may be used to provide an image of the biological sample after it has been stained to visually indicate the presence of a particular biomarker of interest. Some of these methods, such as those disclosed in U.S. patent application Ser. Nos. 09/957,446 and 10/057,729, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that may be deconstructed into its component color parts.

The antibodies used to practice the disclosure are selected to have high specificity for Ras including, for example, mutated Ras. Methods for making antibodies and for selecting appropriate antibodies are known in the art (see, e.g., Celis, ed. (in press) Cell Biology & Laboratory Handbook, 3rd edition (Academic Press, New York)). In some embodiments, commercial antibodies directed to specific Ras proteins may be used to practice the disclosure. The antibodies of the disclosure may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry may be needed to maximize the signal to noise ratio for a particular antibody. Antibody concentrations that maximize specific binding to Ras and minimize non-specific binding (or background) can be determined. In particular embodiments, appropriate antibody titers for use in cytology preparations are determined by initially testing various antibody dilutions on formalin-fixed paraffin-embedded normal and high-grade cervical disease tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody may be then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the disclosure will vary depending on such factors as time for binding, level of specificity of the antibody for Ras protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a biomarker of the present disclosure.

One skilled in the art will know many other suitable carriers for binding antibody or antigen, and will be able to adapt such support for use with the present disclosure. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies.

The present disclosure also includes methods for fixing cells and tissue samples for analysis. Generally, neutral buffered formalin may be used. Any concentration of neutral buffered formalin that can fix tissue or cell samples without disrupting the epitope can be used. In one embodiment a solution of about 10 percent may be used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na3VO4 can be used. In one method a tissue sample or tumor biopsy may be removed from a patient and immediately immersed in a fixative solution which can and preferably does contain one or more phosphatase inhibitors, such as NaF and/or Na3VO4. Preferably, when sodium orthovanadate is used it is used in an activated or depolymerized form to optimize its activity.

Depolymerization can be accomplished by raising the pH of its solution to about 10 and boiling for about 10 minutes. The phosphatase inhibitors can be dissolved in the fixative just prior to use in order to preserve their activity.

Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks they can be analyzed histochemically for virtually any antigen that is stable to the fixing process.

In preferred embodiments, Ras staining may be detected, measured and quantitated automatically using automated image analysis equipment. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. Using such equipment the quantity of the target epitope in unknown cell samples can be determined using any of a variety of methods that are known in the art. The cell pellets can be analyzed by eye such that the optical density reading of the control cells can be correlated to a manual score such as 0, 1⁺, 2⁺ or 3⁺, as in Table 1 below which shows the correlation between quantitative image analysis data measured in optical density (OD) and manual score.

Automated (computer-aided) image analysis systems known in the art can augment visual examination of biological samples. In a representative system, the cell or tissue sample may be exposed to detectably labeled reagents specific for Ras (e.g., mutated Ras), and the magnified image of the cell may be then processed by a computer that receives the image from a charge-coupled device (CCD) or camera such as a television camera. Such a system can be used, for example, to detect and measure expression and activation levels of Her1, pHER1 HER2, HER3, and pERK in a sample. Additional biomarkers are also contemplated by this disclosure. This methodology provides more accurate diagnosis of cancer and a better characterization of gene expression in histologically identified cancer cells, most particularly with regard to expression of tumor marker genes or genes known to be expressed in particular cancer types and subtypes (i.e., different degrees of malignancy). This information permits a more informed and effective regimen of therapy to be administered, because drugs with clinical efficacy for certain tumor types or subtypes can be administered to patients whose cells are so identified.

For example, expression and activation of Ras proteins expressed from tumor-related genes can be detected and quantitated using methods of the present disclosure. Further, expression and activation of proteins that are cellular components of a tumor-related signaling pathway can be detected and quantitated using methods of the present disclosure. Further, proteins associated with cancer can be quantified by image analysis using a suitable primary antibody against biomarkers, such as, but not limited to, Her-1, Her-2, p-Her-1, Her-3, or p-ERK, and a secondary antibody (such as rabbit anti-mouse IgG when using mouse primary antibodies) and/or a tertiary avidin (or Strepavidin) biotin complex (“ABC”).

In practicing the method of the present disclosure, staining procedures can be carried out by a technician in the laboratory. Alternatively, the staining procedures can be carried out using automated systems. In either case, staining procedures for use according to the methods of this disclosure are performed according to standard techniques and protocols well-established in the art.

The amount of Ras can then be quantitated by the average optical density of the stained antigens. Also, the proportion or percentage of total tissue area stained may be readily calculated, as the area stained above an antibody threshold level in the second image. Following visualization of nuclei containing Ras, the percentage or amount of such cells in tissue derived from patients after treatment may be compared to the percentage or amount of such cells in untreated tissue or said tissue prior to treatment.

Pharmaceutical formulations comprising one or more methyltransferase inhibitors and one or more receptor tyrosine kinase inhibitors are provided. Formulations of one or more methyltransferase inhibitors and one or more receptor tyrosine kinase inhibitors may be prepared for storage by mixing with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Alternatively, or additionally, the composition may further comprise a chemotherapeutic agent, cytotoxic agent, cytokine, growth-inhibitory agent, anti-hormonal agent, anti-angiogenic agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (e.g., poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPO™ (injectable microspheres composed of lactic acid-glycolic acid copolymer and leuprolide acetate), and poly-D-(−)-3-hydroxybutyric acid.

Preferably the formulations to be used for in vivo administration are sterile. This may be accomplished by filtration through sterile filtration membranes.

EGFR-mediated disease for treatment may be cancer and may be selected from glioblastoma, head and neck cancer, pancreatic cancer, lung cancer, cancer of the nervous system, gastrointestinal cancer, prostate cancer, ovarian cancer, breast cancer, kidney cancer, retina cancer, skin cancer, liver cancer, genital -urinary cancer, and bladder cancer. In a particular aspect, the EGFR-mediated cancer is lung adenocarcinoma, lung squamous cell carcinoma or non-small cell lung cancer.

In certain embodiments, a composition comprising one or more tyrosine kinase inhibitors and one or more methyltransferase inhibitors may be administered to the patient. In a preferred embodiment, a cytotoxic agent (including for example, maytansinoids, calicheamicins, ribonucleases, and DNA endonucleases) targets or interferes with nucleic acid in the cancer cell. In another embodiment a cytotoxic agent (e.g., taxanes or epothilones) may target or interfere with microtubules and microtubule-dependent mitosis in the cancer cell.

A composition comprising one or more tyrosine kinase inhibitors and one or more methyltransferase inhibitors may be administered to a human patient in accordance with known methods, such as intravenous administration, e.g., as a bolus or by continuous infusion over a period of time, by intramuscular, intraperitoneal, intracerebrospinal, subcutaneous, intra-articular, intrasynovial, intrathecal, oral, topical, or inhalation routes. Intravenous, intraperitoneal, or subcutaneous administration of the antibody is preferred, with subcutaneous or intraperitoneal routes being particular preferred. A preferred administration schedule may be a single dose for an acute disorder or about once every three to four weeks for a chronic disorder, depending on the particular mammal being treated, the type of antibody, and other factors well known to the practitioner. However, other administration schedules are operable herein.

Administration of pharmaceutical compositions according to the disclosure may be through several routes of administration, for example, lingual, sublingual, buccal, in the mouth, oral, in the stomach and intestine, nasal, pulmonary, for example, through the bron chioles and alveoli or a combination thereof, epidermal, dermal, transdermal, vaginal, rectal, ocular, for examples through the conjunctive, uretal, and parenteral to patients in need of such a treatment.

Other therapeutic regimens may be combined with the administration of the composition comprising one or more tyrosine kinase inhibitors and one or more methyltransferase inhibitors. A combined administration includes co-administration, using separate formulations or a single pharmaceutical formulation, and consecutive administration in either order, wherein preferably there may be a time period while both (or all) active agents simultaneously exert their biological activities.

In one embodiment, the treatment of the present disclosure involves the combined administration of a composition comprising one or more tyrosine kinase inhibitors and one or more methyltransferase inhibitors and one or more regulators of immune function in a mammal, such as cytokines, as well as chemotherapeutic agents or growth-inhibitory agents, including co-administration of cocktails of different chemotherapeutic agents. Preferred chemotherapeutic agents include taxanes (such as paclitaxel and docetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service, Ed., M. C. Perry, Williams & Wilkins, Baltimore, Md. (1992).

Available additional treatments for cancer that may be advantageously employed in combination with the therapies and compositions disclosed herein include, without limitation, surgery, radiation therapy, chemotherapy, high dose chemotherapy with stem cell transplant; hormone therapy, and monoclonal antibody therapy.

Different chemotherapeutic agents are known in the art for treating cancer. Cytoxic agents used for treating cancer include doxorubicin, cyclophosphamide, methotrexate, 5-fluorouracil, mitomycin C, mitoxantrone, paclitaxel, taxane formulations such as by way of example only, Abraxane® (ABI-007), Paclitaxel-Cremophor EL, Paclitaxel poliglumex, and Paclitaxel injectable emulsion (PIE), gemcitabine, docetaxel, capecitabine and epirubicin.

Other chemotherapy against cancer includes treatment with one or more of bendamustine, carboplatin (for example, Paraplatin®), carmustine (for example, BCNU®), chlorambucil (for example, Leukeran®), cisplatin (for example, Platinol®), cyclophosphamide injection (for example, Cytoxan®), oral cyclophosphamide (for example, Cytoxan®), dacarbazine (for example, DTIC®), ifosfamide (for example, Ifex®), lomustine (for example, CCNU®), mechlorethamine (for example, nitrogen mustard, Mustargen®), melphalan (for example, Alkeran®), procarbazine (for example, Matulane®), bleomycin (for example, Blenoxane®), doxorubicin (for example, Adriamycin®, Rubex®), epirubicin, Idarubicin (for example, Idamycin®), mitoxantrone (for example, Novantrone®), gemcitabine (for example, Gemzar®), oral mercaptopurine (for example, Purinethol®). methotrexate, pentostatin IV (for example, Nipent®), oral thioguanine (for example, Lanvis®), oral etoposide (for example, VP-16, VePesid®, Etopophos)—etoposide IV (for example, VP-16, VePesid®, Etopophos), vinblastine (for example, Velban®), vincristine (for example, Oncovin®), vinorelbine (for example, Navelbine®), dexamethasone (for example, Decadron®), methylprednisolone (for example, Medrol®), and prednisone (for example, Deltasone®). Erlotinib in combination with gemcitabine is indicated for the treatment of advanced pancreatic cancer.

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies may be given by infusion. They may be used alone or to carry drugs, toxins, or radioactive material directly to cancer cells. Monoclonal antibodies are also used in combination with chemotherapy as adjuvant therapy.

For the prevention or treatment of disease, the appropriate dosage of antibody will depend on the type of disease to be treated, as defined above, the severity and course of the disease, whether the antibody may be administered for preventive or therapeutic purposes, previous therapy, the patient's clinical history and response to the antibody, and the discretion of the attending physician. The antibody may be suitably administered to the patient at one time or over a series of treatments. Depending on the type and severity of the disease, about 1 μg/kg to 15 mg/kg (e.g. 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment may be sustained until a desired suppression of disease symptoms occurs.

The preferred dosage of a composition comprising one or more tyrosine kinase inhibitors and one or more methyltransferase inhibitors may be in the range from about 0.05 mg/kg to about 10 mg/kg. Thus, one or more doses of about 0.3 mg/kg, 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses, of the composition comprising one or more tyrosine kinase inhibitors and one or more methyltransferase inhibitors). An initial higher loading dose, followed by one or more lower doses, may be administered. An exemplary dosing regimen comprises administering an initial loading dose of about 4 mg/kg, followed by a weekly maintenance dose of about 2 mg/kg of the composition comprising one or more tyrosine kinase inhibitors and one or more methyltransferase inhibitors. However, other dosage regimens may be useful. The progress of this therapy may be easily monitored by conventional techniques and assays.

Alternatively, a composition comprising one or more tyrosine kinase inhibitors and one or more methyltransferase inhibitors may be suitably administered serially or in combination with radiological treatments (e.g., irradiation or introduction of radioactive substances—such as those referred to in UICC (Ed.), Klinische Onkologie, Springer-Verlag (1982)).

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the combinations may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the combinations may be given continuously or temporarily suspended for a certain length of time (i.e., a “drug holiday”).

Once improvement of the patient's conditions has occurred, a maintenance dose may be administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

As described herein, the compositions and methods for using the composition comprising a combination of an EGFR inhibitor and a methyltransferase inhibitor, may be formulated without carriers or excipients or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to about 5% of suspending agent, syrups containing, for example, from about 10 to about 50% of sugar, and elixirs containing, for example, from about 20 to about 50% ethanol, and the like. Such pharmaceutical preparations may contain, for example, from about 0.05 up to about 90% of the active ingredient in combination with the carrier, more usually between about 5% and about 60% by weight.

It is understood, however, that a specific dose level for any particular patient will depend upon a variety of factors such as, for example, decreases in the liver and kidney function.

Treatment dosages generally may be titrated to optimize safety and efficacy. Typically, dosage-effect relationships from in vitro studies initially can provide useful guidance on the proper doses for patient administration. Studies in animal models also generally may be used for guidance regarding effective dosages for treatment of cancers in accordance with the present disclosure. In terms of treatment protocols, it should be appreciated that the dosage to be administered will depend on several factors, including the particular agent that is administered, the route administered, the condition of the particular patient, etc. Determination of these parameters are well within the skill of the art. These considerations, as well as effective formulations and administration procedures are well known in the art.

Oral formulations containing the active combinations described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

As described herein, the combination regimen can be given simultaneously or can be given in a staggered regimen, with a 1,2-diphenylpyrrole derivative being given at a different time during the course of chemotherapy than an EGFR inhibitor. This time differential may range from several minutes, hours, days, weeks, or longer between administration of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

in other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action, such as other cholinergic agents, other serotoninergic agents, alpha adrenergic agents, CCK_{A} antagonists, 5-HT₃ antagonists, NMDA receptor antagonists, opioids, prokinetics, tachykinins, antalarmin, and Z-338.

In some embodiments, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In other embodiments, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, the pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

In other embodiments, the pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

In some embodiments, the pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In other embodiments, the pharmaceutical compositions provided herein in a modified release dosage form may be prepared by methods known to those skilled in the art, including direct compression, dry or wet granulation followed by compression, melt-granulation followed by compression.

In some embodiments, the pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

In some embodiments, the pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

In some embodiments, the pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

In some embodiments, the pharmaceutical compositions provided herein in an immediate release dosage form are capable of releasing not less than 75% of the therapeutically active ingredient or combination and/or meet the disintegration or dissolution requirements for immediate release tablets of the particular therapeutic agents or combination included in the tablet core, as set forth in USP XXII 1990 (The United States Pharmacopeia.)

In other embodiments, the pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

In further embodiments, the pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

In some embodiments, the pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

In another embodiment, the pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

In one embodiment, the pharmaceutical compositions provided herein for topical administration may be formulated to be immediate release or modified release, including delayed-sustained-, pulsed-, controlled-, targeted, and programmed release.

In another embodiment of the disclosure, an article of manufacture containing materials useful for the treatment of the diseases or disorders described above is provided. The article of manufacture may comprise a container and a label or package insert on or associated with the container. Suitable containers include, for example, bottles, vials or syringes. The containers may be formed from a variety of materials such as glass or plastic. The container holds a composition that may be effective for treating the condition and may have a sterile access port (e.g., the container may be an intravenous solution bag or a vial having a stopper pierceable by a hypodermic injection needle). At least two active agents in the composition may be one or more methyltransferase inhibitors, such as Methotrexate and one or more tyrosine kinase inhibitors. The label or package insert may indicate that the composition may be used for treating the condition of choice, such as cancer.

Moreover, the article of manufacture may comprise (a) a first container with a composition contained therein, wherein the composition comprises one or more methyltransferase inhibitors, such as methotrexate, and (b) a second container with a composition contained therein, wherein the composition comprises one or more receptor tyrosine kinase inhibitors. The article of manufacture in this embodiment of the disclosure may further comprise a package insert indicating that the first and second compositions can be used in combination to treat a disease or disorder including, for example, cancer. Additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Overcoming KRAS Mutant Resistance to Receptor Tyrosine Kinase Inhibitors

The resistance of KRAS mutant cells (e.g., activating RAS mutations) to treatment with a receptor tyrosine kinase inhibitor may be overcome by administering to the cells an inhibitor of the dihydrofolate reductase (DHFR) pathway. Such an effect can be greatened by co-administering to the cells a receptor tyrosine kinase inhibitor.

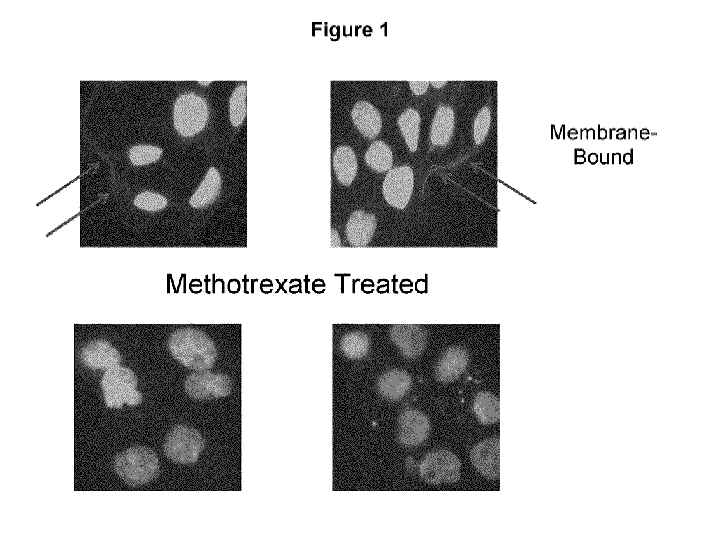

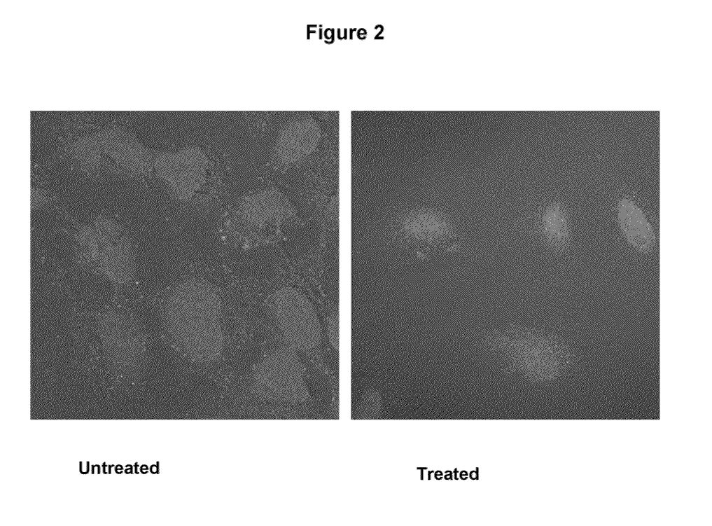

In an exemplary method, the A549 lung tumor cell line was treated for 48 hours with 1.0 micromolar Methotrexate followed by immunocytochemistry staining for RAS on slides. As shown in FIGS. 1 and 2, localization of mutant Ras in untreated cells is diffuse and at the membrane. Upon treatment of the cells with Methotrexate, overall abundance of mutant Ras is diminished as well as mislocalization from the plasma membrane resulting in highly cytoplasmic staining of mutant Ras.

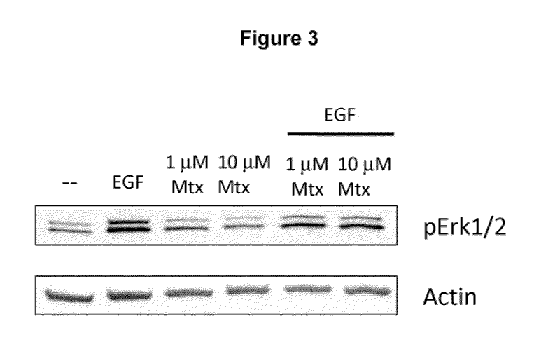

Additionally, in vitro cell signaling studies were conduced to examine MAPK signaling in RAS mutant cells treated with Methotrexate. For Methotrexate treatments, A549 lung tumor cells were treated with 1.0 or 10.0 μM Methotrexate in full serum media for 24 hours followed by withdrawal of Methotrexate and serum starvation (1.0% FBS) for 48 hours in the absence or presence of 10 ng/ml EGF added for the final 10 minutes before harvest of the cells. Next, whole cell extracts were isolated for Western blot analysis of pErk1/2 and pAkt. The presence of β-actin served as an internal control for loading differences between lanes. Notably, as little as 1.0μM Methotrexate for 24 hours reduced both basal and EGF-induce pErk1/2 levels in A549 cells (see, FIG. 3).

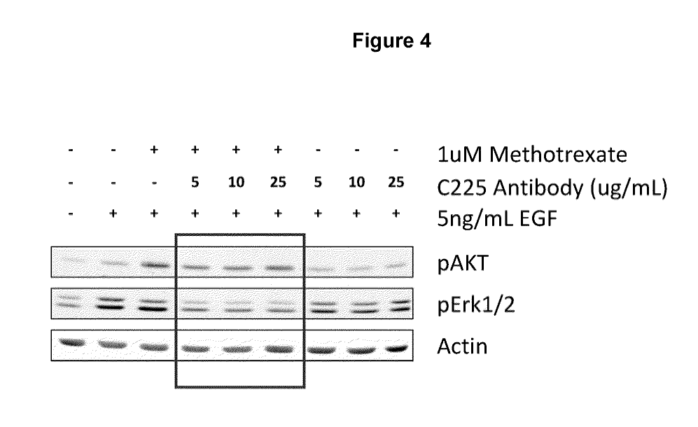

Subsequently, KRAS mutant A549 cells were treated with EGF, Methotrexate, C225 (an anti-EGFR antibody), or a combination of EGF, Methotrexate and C225 in the context of EGF stimulation. For Methotrexate treatments, A549 lung tumor cells were treated with 1.0 μM Methotrexate in full serum media for 24 hours followed by withdrawal of Methotrexate and serum starvation (1.0% FBS) for 48 hours in the absence or presence of 5 ng/ml EGF added for the final 10 minutes before harvest of the cells. Treatment of the cells with Methotrexate or C225 antibody by themselves resulted in a slight reduction in the amount of detectable pAKT and pErk1/2. As expected, EGF stimulation of A549 cells potently activated the MAPK pathway as shown by pErk1/2 induction. Alone 1.0 μM Methotrexate or increasing amounts of C225 antibody (e.g., EGFR antibody) up to 25 μg/ml only slightly inhibited pErk1/2. Unexpectedly, cells treated with Methotrexate in combination with as little as 1.0 μg/ml C225 antibody exhibited a synergistic effect (an effect that was much greater than the additive effects of Methotrexate and C225 taken alone) on reducing MAPK signaling as evidenced by reduced detection of pAKT and pErk 1/2 (see, FIG. 4). Therefore, the inventor has discovered that co-administration of Methotrexate with an anti-EGFR antibody is able to overcome the effects of MAPK signaling activation in cells that harbor mutations in RAS.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for sensitizing and treating a cell harboring a Ras mutation, the method comprising,
contacting the cell with a therapeutically effective amount of one or more inhibitors of the dihydrofolate reductase (DHFR) pathway; and
contacting the cell with a therapeutically effective amount of one or more receptor tyrosine kinase inhibitors.

2. The method of claim 1, wherein the inhibitor of the DHFR pathway is a methyltransferase inhibitor.

3. The method of claim 2, wherein the methyltransferase inhibitor is Methotrexate.

4. The method of claim 1, wherein Ras is k-Ras (SEQ ID NO: 1), n-Ras (SEQ ID NO: 2) or h-Ras (SEQ ID NO: 3).

5. The method of claim 4, wherein the k-Ras mutations are at position 12, 13 or 61.

6. The method of claim 5, wherein the k-Ras mutations are selected from the group consisting of: G12A, G12N, G12R, G12C, G12S, G12V, G13N and Q61H.

7. The method of claim 4, wherein the h-Ras or n-Ras mutations are at positions 12, 13 or 61.

8. The method of claim 1, wherein the receptor tyrosine kinase inhibitor is an antibody.

9. The method of claim 1, wherein the tyrosine kinase inhibitor targets HER1 (EGFR), HER2/neu, HER3, or any combination thereof.

10. The method of claim 8, wherein the antibody is a monoclonal antibody.

11. The method of claim 10, wherein the monoclonal antibody is cetuximab (Erbitux), panitumumab, zalutumumab, nimotuzumab or matuzumab.

12. The method of claim 1, wherein the receptor tyrosine kinase inhibitor is a small molecule inhibitor.

13. The method of claim 12, wherein the small molecule inhibitor is gefitinib, erlotinib or lapatinib.

14. A method for treating a disease or disorder in a subject, the method comprising, administering to the subject a therapeutically effective amount of one or more receptor tyrosine kinase inhibitors and a therapeutically effective amount of one or more inhibitors of the dihydrofolate reductase (DHFR) pathway.

15. The method of claim 14, wherein the inhibitor of the DHFR pathway is a methyltransferase inhibitor.

16. The method of claim 15, wherein the methyltransferase inhibitor is Methotrexate.

17. The method of claim 14, wherein the disease or disorder is characterized by one or more Ras mutations.

18. The method of claim 17, wherein Ras is k-Ras (SEQ ID NO: 1), n-Ras (SEQ ID NO: 2) or h-Ras (SEQ ID NO: 3).

19. The method of claim 18, wherein the k-Ras mutations are at position 12, 13 or 61.

20. The method of claim 19, wherein the k-Ras mutations are selected from the group consisting of: G12A, G12N, G12R, G12C, G125, G12V, G13N and Q61H.

21. The method of claim 18, wherein the h-Ras or n-Ras mutations are at positions 12, 13 or 61.

22. The method of claim 14, wherein the receptor tyrosine kinase inhibitor is an antibody.

23. The method of claim 14, wherein the tyrosine kinase inhibitor targets HER1 (EGFR), HER2/neu, HER3, or any combination thereof.

24. The method of claim 22, wherein the antibody is a monoclonal antibody.

25. The method of claim 24, wherein the monoclonal antibody is cetuximab (Erbitux), panitumumab, zalutumumab, nimotuzumab or matuzumab.

26. The method of claim 14, wherein the receptor tyrosine kinase inhibitor is a small molecule inhibitor.

27. The method of claim 26, wherein the small molecule inhibitor is gefitinib, erlotinib or lapatinib.

28. The method of claim 14, wherein the therapeutically effective amount of one or more tyrosine kinase inhibitors and the therapeutically effective amount of one or more methyl-transferase inhibitors are optionally adapted for a co-treatment with radiotherapy or radio-immunotherapy.

29. The method of claim 14, wherein the disease or disorder is cancer.

30. The method of claim 29, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

31. The method of claim 14, wherein the subject is a cancer patient.

32. A method for treating a subject with a disease or disorder, the method comprising:
a. obtaining a biological sample from the subject;
b. assaying the biological sample for one or more Ras mutations;
c. determining if one or more Ras mutations are present in the biological sample; and
d. administering to the subject one or more tyrosine kinase inhibitors and one or more inhibitors of the DHFR pathway where one or more Ras mutations are present in the biological sample and administering to the subject one or more tyrosine kinase inhibitors where no Ras mutations are present in the biological sample.

33. The method of claim 32, wherein the inhibitor of the DHFR pathway is a methyltransferase inhibitor.

34. The method of claim 33, wherein the methyltransferase inhibitor is Methotrexate.

35. The method of claim 32, wherein the disease or disorder is characterized by one or more Ras mutations.

36. The method of claim 35, wherein Ras is k-Ras (SEQ ID NO: 1), n-Ras (SEQ ID NO: 2) or h-Ras (SEQ ID NO: 3).

37. The method of claim 36, wherein the k-Ras mutations are at position 12, 13 or 61.

38. The method of claim 37, wherein the k-Ras mutations are selected from the group consisting of: G12A, G12N, G12R, G12C, G12S, G12V, G13N and Q61H.

39. The method of claim 36, wherein the h-Ras or n-Ras mutations are at positions 12, 13 or 61.

40. The method of claim 32, wherein the receptor tyrosine kinase inhibitor is an antibody.

41. The method of claim 40, wherein the antibody is a monoclonal antibody.

42. The method of claim 41, wherein the monoclonal antibody is cetuximab (Erbitux), panitumumab zalutumumab, nimotuzumab or matuzumab.

43. The method of claim 32, wherein the receptor tyrosine kinase inhibitor is a small molecule inhibitor.

44. The method of claim 43, wherein the small molecule inhibitor is gefitinib, erlotinib or lapatinib.

45. The method of claim 32, wherein the therapeutically effective amount of one or more tyrosine kinase inhibitors and the therapeutically effective amount of one or more methyl-transferase inhibitors are optionally adapted for a co-treatment with radiotherapy or radio-immunotherapy.

46. The method of claim 32, wherein the disease or disorder is cancer.

47. The method of claim 46, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

48. The method of claim 32, wherein the subject is a cancer patient.

49. The method of claim 32, wherein the biological sample is assayed for Ras mutations by analyzing nucleic acid obtained from the sample.

50. The method of claim 32, wherein the biological sample is assayed for Ras mutations by analyzing proteins obtained from the sample.

51. The method of claim 32, wherein biological sample is a tumor biopsy.

52. The method of claim 32, wherein the biological sample is an aspirate.

53. The method of claim 32, wherein the tyrosine kinase inhibitor targets HER1 (EGFR), HER2/neu, HER3, or any combination thereof.

54. A pharmaceutical composition comprising a therapeutically effective amount of one or more tyrosine kinase inhibitors and a therapeutically effective amount of one or more inhibitors of the DHFR pathway.

55. The pharmaceutical composition of claim 54, wherein the inhibitor of the DHFR pathway is a methyltransferase inhibitor.

56. The pharmaceutical composition of claim 55, wherein the methyltransferase inhibitor is Methotrexate.

57. The pharmaceutical composition of claim 54, wherein the receptor tyrosine kinase inhibitor is an antibody.

58. The pharmaceutical composition of claim 57, wherein the receptor tyrosine kinase inhibitor targets HER1 (EGFR), HER2/neu, HER3, or any combination thereof.

59. The pharmaceutical composition of claim 57, wherein the antibody is a monoclonal antibody.

60. The pharmaceutical composition of claim 59, wherein the monoclonal antibody is cetuximab (Erbitux), panitumumab zalutumumab, nimotuzumab or matuzumab.

61. The pharmaceutical composition of claim 54, wherein the receptor tyrosine kinase inhibitor is a small molecule inhibitor.

62. The pharmaceutical composition of claim 61, wherein the small molecule inhibitor is gefitinib, erlotinib or lapatinib.


SEQUENCE LISTING (3 sequences)

SEQ ID NO: 1 - protein, 188 aa, human
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 2 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 3 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS